(19) United States
(12) Patent Application Publication
(10) Pub. No.: US 2006/0259074 A1
(43) Pub. Date: Nov. 16, 2006

(54) Methods and devices for anchoring to soft tissue

(76) Inventors: Brian Kelleher, San Diego, CA (US); Matt Yurek, San Diego, CA (US)

Correspondence Address: SCOTT EVANS, 1252 COUNTRY HILLS DR., SANTA ANA, CA, 92705, US

(21) Appl. No.: 11/356,949

(22) Filed: Feb. 18, 2006

Related U.S. Application Data
(60) Provisional application No. 60/659,445, filed on Feb. 22, 2005.

(51) Int. Cl. A61B 17/08

(52) U.S. Cl. 606/213

(57) ABSTRACT

Disclosed are methods and devices for placing anchors into soft tissue such as the walls of the stomach. The anchoring device includes a tissue interface that is introduced in a collapsed configuration and which expands radially outward. The tissue interface includes a framework capable of distributing a load applied to the linkage element across the surface area of the tissue interface.

### CLAIM OF PRIORITY

This application claims priority under 35 U.S.C. §119(e) to U.S. Provisional Application No. 60/659,445, filed Feb. 22, 2005, the entire contents of which are hereby expressly incorporated by reference.

### BACKGROUND

1. Field of the Invention

The present invention relates to devices and methods for attaching soft tissues and in particular, to novel tissue anchoring elements.

2. Description of the Related Art

The securement of soft tissue segments has traditionally been done using suturing or stapling devices. However, when segments of tissue are attached together and then exposed to tension post-operatively, such techniques often do not hold up over time. For example, when two segments of the stomach are sewn together the sutures that hold the segments together are in tension post-operatively. In order to prevent the sutures from pulling through the stomach wall over time, the sites where the sutures puncture the outer wall of the stomach are sometimes reinforced with sections of tear-resistant material, called pledgets.

The use of pledgets is not always possible especially when securing the wall of an organ that has a surface not easily accessible during the procedure. As an example, when performing an endoluminal gastroplasty procedure, that is, when sewing the wall of the stomach to itself from within the lumen of the stomach, only the inner wall is accessible. Sutures that are placed through the wall can be strain-relieved with a pledget or similar device only along the inner surface of the wall, but not along the outer wall (unless a pledget or similar device is passed through the wall, which is generally not practical). When sutures placed in this way are exposed to tension, as is the case when a gastroplasty procedure is done to create a gastric restriction, the sutures generally pull out over time.

Similarly, when attaching a foreign body to a segment of soft tissue using attachment techniques such as suturing, if the foreign body is subjected to forces postoperatively, the foreign body will typically pull loose from the tissue segment.

There is therefore a need for robust tissue securement devices and methods that enable tissue-to-tissue attachment and attachment of foreign bodies to tissue with reduced chance of detachment occurring post-operatively if the securement device is placed under tension. More specifically, there is a need for robust tissue securement devices which can be delivered endoscopically, as through a rigid endoscope, or endoluminally, as through a flexible endoscope.

### BRIEF SUMMARY OF THE INVENTION

The preferred methods and devices described herein provide for improved methods and devices for tissue fastening, and, in particular, to soft tissue anchoring elements and deployment thereof.

In the preferred embodiments, the anchoring elements can be delivered endoscopically, as through a rigid endoscope, or endoluminally, as through a flexible endoscope. The anchors are designed to collapse to a small diameter so that they can be placed into the working channel of an endoscope and then expand to a larger diameter upon deployment. The expanded diameter presents a large surface area and is designed such that any force applied to the anchor is distributed to this surface area and the resultant force per unit area is reduced as compared to sutures or staples. The resultant force per unit area is intended to be small enough to prevent pull-out of the anchor through the soft tissue wall.

In several preferred embodiments, the anchors have a proximal portion and a distal portion and, after deployment through the soft tissue wall, at least a part of the proximal portion resides along the inner soft tissue wall and at least a part of the distal portion resides along the outer soft tissue wall. Furthermore, the anchor is capable of transferring a force applied to the proximal portion through the soft tissue wall to the distal portion. Once deployed, the diameter of the distal portion expands to a new diameter that is greater than the collapsed diameter of the anchor.

The soft tissue anchors can also be constructed from materials or designs that promote cellular ingrowth. The ingrowth into the distal surface secures the distal surface to the tissue and is intended to lock the distal portion to the tissue wall to resist pull out of the anchor. Cellular ingrowth can be promoted by several factors such as the selection of biocompatible materials, designing material surfaces that encourage cellular migration, through the use of growth promoting pharmaceuticals, and by applying pressure on the outer soft tissue wall with the distal portion of the expanded anchor at least until cellular ingrowth occurs.

In a preferred embodiment of the present invention, a tissue securement device comprises a tissue-penetrating device, an anchor element and a linkage element. The tissue-penetrating device is deployed at an initial point of securement at least partially through the target tissue mass. The tissue-penetrating device may be an independent element, or it may be part of the anchor element, or it may be part of a delivery system for the anchor element. The preferred embodiment may utilize devices and methods for isolating the potential treatment site whereby soft tissue is aspirated into a target vessel to isolate the potential treatment site from surrounding structures. Once the target tissue has been isolated from surrounding tissue or organs, the anchor element is deployed. The anchor element preferably incorporates spreading elements to engage a region of tissue wider than the diameter of the tissue-penetrating device. A linkage element may be attached to the anchor element which can serve as a secondary attachment point for other devices, systems or methods. The secondary point of securement may be associated with another tissue segment, or may be associated with a foreign body or another anchor device.

In a preferred embodiment of the invention, the anchor element consists of elements that are deployed from, or are part of, the tissue-penetrating device and which consist of a collapsed umbrella structure that is inserted through the wall of the soft tissue and deployed on the other side. The umbrella unfolds once placement is complete. The position of the umbrella is secured to the outer wall of the soft tissue with one or more fastening members that are deployed by the tissue-penetrating device and traverse the soft tissue wall with anchor points on both sides of the tissue wall. A linkage element is connected to the umbrella center, traverses the tissue wall and remains along the inside wall of tissue.

Another feature of the invention is that the anchor element may be placed at various locations in or around the soft tissue wall. The soft tissue of the stomach wall, by example only, is comprised of several layers of tissue including mucosa, sub-mucosa, muscle and serosa. One anchor placement method may utilize the whole tissue wall whereby at least part of the anchor may be placed through the wall and reside against the serosa. Another anchor placement method may utilize an anchor system whereby at least part of the anchor is placed in the sub-mucosa. Yet another anchor placement method may utilize an anchor system whereby at least part of the anchor is placed in the muscle. In all embodiments described, although one particular anchor location may be detailed, it is anticipated that the anchor may be positioned in other layers of soft tissue and that other anchor sites other than the ones detailed may be utilized.

In another embodiment of the invention, the anchor element is formed by two parallel umbrella structures that are connected by a center coil spring. One umbrella is inserted through the soft tissue wall and deployed on the outer wall and the other is deployed on the inner wall so that the soft tissue is sandwiched in between these two umbrella structures. The spring is attached to both umbrellas and maintains tension along their common axis to draw each umbrella together when deployed. The position of the umbrellas can also be secured to the outer and inner walls of the soft tissue with one or more fastening members that are deployed by the tissue-penetrating device or other means and traverse the soft tissue wall and the umbrella structure with anchor points on both sides of the tissue wall.

In another embodiment of the invention, the anchor element is comprised of a piece of mesh sheet to which is attached a linkage element. Two adjacent folds of soft tissue wall are brought together and the mesh is placed along the innermost fold. The mesh is secured in position by positioning fastening members through one tissue fold, the mesh and then the next tissue fold. The mesh can also be secured in position by placing fastening bands around the folded tissue and mesh.

In another embodiment of the invention, the anchor element is comprised of a braided tubular mesh with a small linkage element attached at one end. The tubular mesh is collapsed and inserted through the wall of a single fold of tissue and allowed to expand on the other side. The mesh can be secured in position by positioning fastening members or bands through or around the tissue/mesh combination.

In still another embodiment of the invention, the anchor element is comprised of two mesh balloon shaped wireforms that are connected together end to end. A collapsing element is connected to the apex of one of the braided mesh wireforms and extends along the central axis of both wireforms and out the apex of one the second braided mesh wireform. A linkage element is attached to the inner end of this collapsing element.

In another embodiment of the present invention the anchor element is a piece of mesh or pledget-like material that is placed through the soft tissue wall and deployed next to the serosa. The material may utilize supporting elements that assist in deploying the material and which may provide a structure to which fastening members may be attached.

An additional embodiment of the present invention utilizes a piece of mesh material that is deployed and spread out once through the soft tissue. The device utilizes supporting elements that move from a collapsed state to an uncollapsed or deployed state. In the deployed condition the supporting elements lock into position to provide structural integrity to the mesh. The mesh in all these embodiments may promote cell ingrowth or may facilitate attachment of cellular structures once ingrowth occurs.

All of these embodiments are intended to be within the scope of the present invention herein disclosed. These and other embodiments of the present invention will become readily apparent to those skilled in the art from the following detailed description of he preferred embodiments having reference to the attached figures. The invention is not limited to any particular preferred embodiment(s) disclosed.

### BRIEF DESCRIPTION OF THE DRAWINGS

The foregoing aspects and many of the attendant advantages of the invention will become readily appreciated as the same become better understood by reference to the following detailed description, when taken in conjunction with the accompanying drawings, wherein:

FIG. 1a is a schematic view showing a portion of soft tissue drawn into a stabilizing section on the end of an endoscope;

FIG. 1b is a section view of the stabilizing section of FIG. 1 illustrating an alternative embodiment with optional fastening channels;

FIG. 2a is a schematic view of an embodiment of a collapsed everted umbrella inside a tissue-penetrating device with the stabilizing section removed;

FIG. 2b is a schematic view of the everted umbrella of FIG. 2a partially deployed through a soft tissue wall;

FIG. 3 is a schematic view of the everted umbrella of FIG. 2b fully deployed;

FIG. 4 is a schematic view of the everted umbrella of FIG. 3 fully deployed showing an alternate design utilizing fastening members;

FIG. 5 is a section view of an everted umbrella inside an alternate embodiment of a stabilizing section of FIG. 1b showing alternate side fastening channels and fastening members;

FIG. 6a is a schematic view of an alternative embodiment of an umbrella collapsed inside a tissue penetrating device;

FIG. 6b is a schematic view of the umbrella of FIG. 6a in a partially deployed position;

FIG. 6c is a schematic view of the umbrella of FIG. 6a in a fully deployed position;

FIG. 7 is a schematic view of an embodiment of a parallel umbrella anchor fully collapsed inside a tissue penetrating device;

FIG. 8a is a schematic view of the parallel umbrella anchor of FIG. 7 deployed through a layer of soft tissue;

FIG. 8b is a schematic side elevation view of the umbrella device of FIG. 8a with an alternate deployment site of the lower umbrella in the submucosa;

FIG. 9a is a schematic view showing folds of soft tissue prior to placement of a mesh anchor;

FIG. 9b is a section view of the folds of soft tissue of FIG. 9a after placement of a mesh anchor and after fastening elements have been deployed;

FIG. 9c is a section view of folds of soft tissue of FIG. 9a after placement of a mesh anchor and a band;

FIG. 10 is a schematic view of an embodiment of a mesh tube anchor and a delivery method;

FIG. 11 is a section view of mesh balloon shaped wireforms collapsed inside a tissue penetrating device prior to placement;

FIG. 12 is a section view of the mesh balloon shaped wireforms of FIG. 11 across soft tissue partially deployed;

FIG. 13a is a section view of the mesh balloon shaped wireforms of FIG. 12 across soft tissue after deployment;

FIG. 13b is a section view of the mesh balloon shaped wireforms of FIG. 13a showing an alternate deployment mechanism;

FIG. 14 is a section view of an alternate embodiment of mesh balloon shaped wireforms shown collapsed inside a tissue penetrating device;

FIG. 15 is a schematic view of the mesh balloon shaped wireforms of FIG. 14 partially deployed through a soft tissue wall;

FIG. 16 is a schematic view of the mesh balloon shaped wireforms of FIG. 15 fully deployed;

FIG. 17a is a schematic view of a mesh anchor shown collapsed inside a tissue penetration device and partially deployed across tissue walls;

FIG. 17b is a section view of the mesh anchor of FIG. 17a shown deployed along the serosa;

FIG. 17c is a schematic view of one embodiment of the mesh anchor rolled up prior to placement;

FIG. 17d is a schematic view of an alternate embodiment of the mesh anchor showing deployment members;

FIG. 17e is a schematic view of an alternate embodiment of the mesh anchor;

FIG. 17f is a schematic view of the mesh anchor of FIG. 17e showing the anchor collapsed for insertion;

FIG. 17g is a schematic view of an alternate embodiment of the mesh anchor showing distending members;

FIG. 17h is a schematic view of the mesh anchor of FIG. 17g showing the distending members partially collapsed for insertion;

FIG. 17i is a schematic view of an alternate embodiment of the mesh anchor showing distending members;

FIG. 17j is a schematic view of the mesh anchor of FIG. 17i showing part of the collapsing mechanism;

FIG. 17k is a schematic view of the mesh anchor of FIG. 17i showing another part of the collapsing mechanism;

FIG. 18 is a schematic view of an alternative embodiment of the mesh anchor showing multiple anchors loaded inside a tissue penetrating device;

FIG. 19 is a schematic view of another embodiment of tissue anchor utilizing a collapsible mesh sail;

FIG. 20 is a schematic view of the collapsible mesh sail of FIG. 19 shown in a deployed configuration;

FIG. 21 is a detailed view of the strut attachment of the collapsible sail of FIG. 19;

FIG. 22 is a detailed view of a the collapsible mesh sail showing a strut base and linkage element:

### DETAILED DESCRIPTION OF THE INVENTION

As has been described, the attachment of fastening devices to soft tissue often depends on the penetration of soft tissue and placement of pledgets, mesh, umbrellas or other anchors on the outer wall of soft tissue. This can be dangerous because other important blood vessels, nerves or organs such as the liver, lungs, heart, gall bladder, kidneys, reproductive organs or other sensitive tissue often reside close to the point of placement, and the exact location of these sensitive structures is rarely known prior to intervention on the soft tissue by the physician. Many of the methods and devices described in this application can be placed with the use of a “safe harbor” delivery system that permits penetration of the soft tissue wall and placement of the anchor on the opposite side with less fear of damage to surrounding sensitive structures.

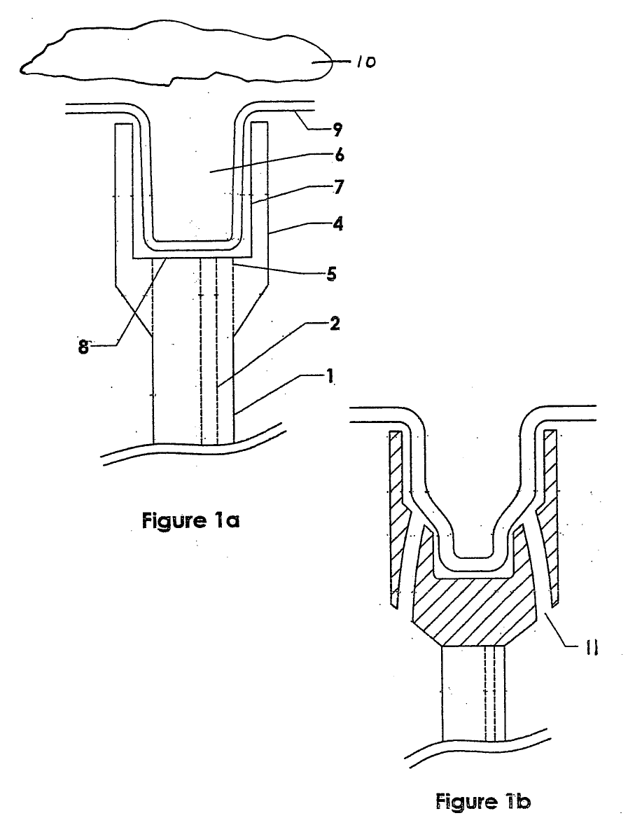

As shown in FIG. 1a, an endoscope 1 with a working channel 2, is employed that has a stabilizing element 4 attached to the distal end of the scope 5. This stabilizing element 4 is sized to be coupled with the distal end of the endoscope 5. The stabilizing element 4 can be coupled to the endoscope 1 prior to placement into the patient's body or the stabilizing element 4 can be coupled to the endoscope after the stabilizing element 4 and the endoscope 1 have been placed inside the patient's body. The stabilizing element 4 can be coupled to the distal end of the endoscope 5 using a friction or press fit, using mechanical attachments or with adhesives. Preferably the stabilizing element 4 can be later de-coupled from the endoscope at the end of the procedure so that the endoscope 1 can be used for other procedures. The distal portion of the stabilizing element 4 has an open cavity 6 that is shaped similar to a cup. The inner portion of this cavity 6 has two sides 7 that are generally parallel to the axis of the endoscope and a bottom surface 8 that is generally perpendicular to the axis of the endoscope. The working channel of the endoscope 2 is in fluid communication with the interior space of the cavity 6. When the stabilizing element 4 is placed against soft tissue 9 and suction is applied through the working channel 2 of an endoscope, negative pressure is created inside of the cavity 6. The negative pressure inside the cavity 6 draws the soft tissue 9 into the cavity 6. A tissue securement system comprising a tissue-penetrating device, an anchoring element and a linkage element can be positioned inside the working channel of an endoscope 1. When the soft tissue 9 is drawn into the cavity 6 it is moved away from sensitive tissue or organs 10 and permits safe penetration of the soft tissue with the tissue-penetrating device. An anchoring element can be safely deployed and the endoscope withdrawn. An alternative embodiment of the stabilizing element 4 is shown in FIG. 1b that utilizes fastener delivery channels 11. These channels 11 join the cup at an angle to the working channel and can facilitate attachment of various fastening elements to the soft tissue 9 at alternate points spaced apart from the working channel 2 of the endoscope 1. These channels 11 may connect to an alternate working channel of an endoscope or they may be accessed individually using separate devices. Although the use of an endoscope is described in this application, it is also anticipated that the delivery of the stabilizing element 4 and various fastening devices could be accomplished without an endoscope or without reliance on the working channel of an endoscope.

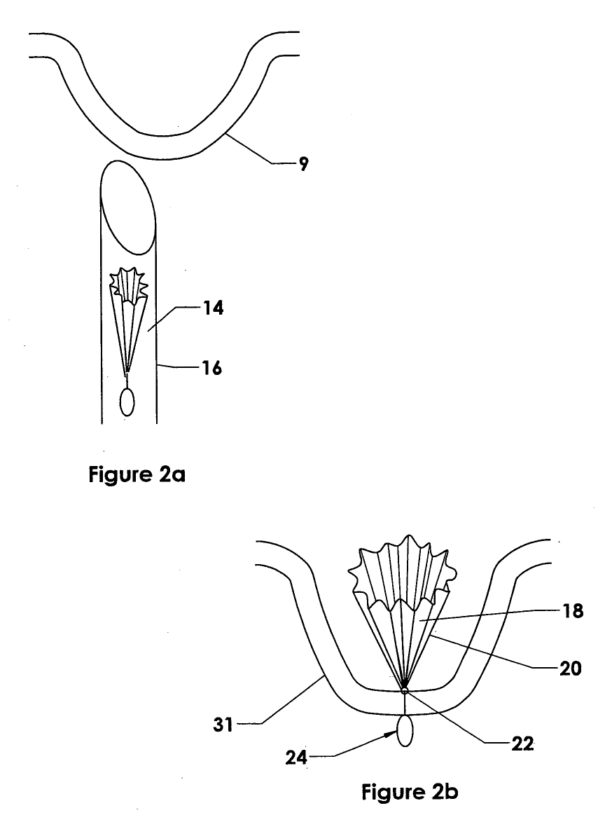

One embodiment of a soft tissue anchor is an everted umbrella. The umbrella 14 is shown in FIG. 2a in a collapsed state positioned inside an anchor delivery device 16. The anchor delivery device 16 may be a hollow needle, a hypodermic tube, a catheter or a sheath. The umbrella 14 is collapsed for delivery through the anchor delivery device 16 and can be pushed out of the anchor delivery device 16 by a pusher rod once the anchor delivery device 16 has been inserted across the soft tissue 9. The umbrella 14 is shown in a partially deployed state in FIG. 2b. The umbrella 14 is constructed of a disk shaped mesh element 18 with supporting struts 20 that are attached to the mesh element and are designed to provide structural rigidity to the umbrella 14.

The various embodiments of mesh elements 18 that are described in this application can be constructed from various materials such as metal, plastic, fabric or wire and may be braided, woven and may be fabricated from a continuous piece of material. Especially effective are materials that promote the ingrowth of surrounding cells. An example of one type of material is Marlex® mesh (Davol, Cranston, R.I.) which stimulates increased tissue fixation and is designed to bond firmly to a host facia. Alternatively the mesh or material may be coated or impregnated with tissue growth substances such as pharmaceuticals that promote or stimulate tissue growth near the mesh. This tissue growth promotion may accelerate the anchoring process. These types of materials are suitable for all the tissue contact surfaces described herein because such tissue ingrowth will inherently strengthen the securement capabilities of the anchors described. Mesh is suggested in this application because it is lightweight, easily collapsible and can provide a structure to promote ingrowth of cellular materials. However other non-mesh materials may also be suitable. Tissue ingrowth can fill the open mesh structure, strengthen the anchoring capabilities of the device and reduce the chance of cellular irritation.

In another embodiment of the mesh 18, a tissue growth enhancing surface may be incorporated on a single side of the mesh that is intended to be in contact with the soft tissue wall, particularly the serosa. The opposite side of the mesh 18 may incorporate a coating or material that is designed to prevent other cellular structures, tissues or organs from adhering to the mesh. An example of one type of material is Dualmesh® (WL Gore, Flagstaff, Ariz.). This may be advantageous to prevent adjacent tissue or organs such as the liver, kidney, gall bladder, intestines or reproductive organs from attaching to the mesh anchors when deployed. The struts 20 can be formed out of metal, metal alloys such as Nitinol or Elgiloy or from plastic or plastic alloys. The apex of the umbrella 22 has a linkage element 24 attached that is positioned on the inside of the soft tissue wall 31 when the umbrella is deployed. This linkage element 24 can be used for attaching other devices or can be connected to other linkage elements for secondary interventions or purposes.

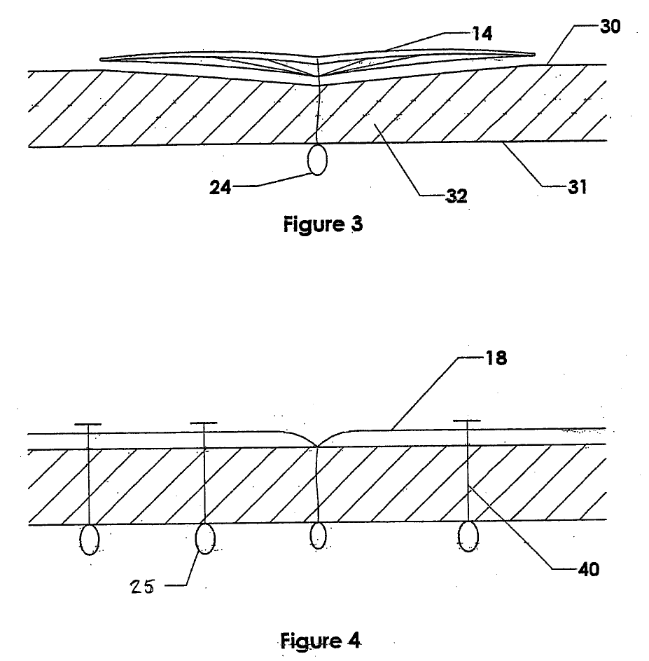

FIG. 3 shows the umbrella 14 fully deployed and spread out along the outer soft tissue wall 30. In this configuration the mesh 18 and the struts 20 are unfurled similar to a flower unfolding. Also in this configuration, the linkage element 24 is shown transversing the soft tissue wall 32 and is exposed on the inside wall 31. In this configuration, any force that is applied to the linkage element 24 is distributed along the struts 20, the mesh 18 and the outer soft tissue wall 30. These struts 20 function to support the mesh 18 and also they distribute any force applied to the linkage element 24 across a large surface area resulting in a lower force per unit area (or pressure) on the surrounding tissue than a single point attachment device. This reduction in pressure is designed to inhibit the migration of the umbrella through the outer soft tissue wall 30 and through the tissue inner wall 32 when tension is applied to the linkage element 24.

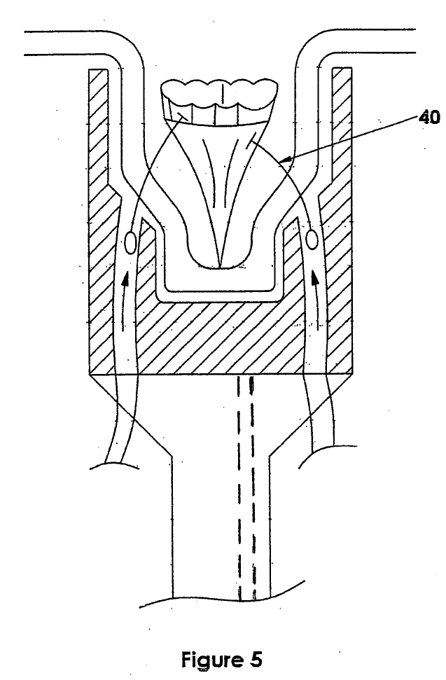

As shown in FIG. 4, the strength of the anchoring device may be further enhanced by the placement of additional fastening elements 40 at various attachment points. These fastening elements 40 are displaced apart from the center of the umbrella and the linkage elements 25. These fastening elements 40 attach the umbrella 14 to the outer wall 30 wherever deployed. These elements 40 may be T-tags or other devices previously described and may include a linkage element 24 (loop, hook, ring, barb or other) at the inner end. These additional fastening elements anchor the umbrella 14 to the soft tissue and also help prevent pullout of the anchoring umbrella 14. In addition to securing the umbrella as described, these fastening elements 40 may also provide a constant pressure between the umbrella 14 and the outer soft tissue wall 30 which may promote cell ingrowth into the mesh 18. These additional fastening elements 40 can be placed by various means such as direct injection with a separate needle or puncture device. However the fastening elements 40 can also be deployed using the fastener delivery channels 11 of the stabilizing element 4 shown in FIG. 1b. As shown in more detail in FIG. 5, these fastener delivery channels 11 angle toward the center of the stabilizing element 4 and can be used to direct an insertion device that is initially parallel to the central axis of the endo scope at an angle as shown by the arrow to facilitate puncture of the tissue wall and umbrella mesh using various fastening devices 40. Alternatively the fastening elements 40 may be placed directly through the soft tissue and the umbrella using standard anchor placement techniques known in the art.

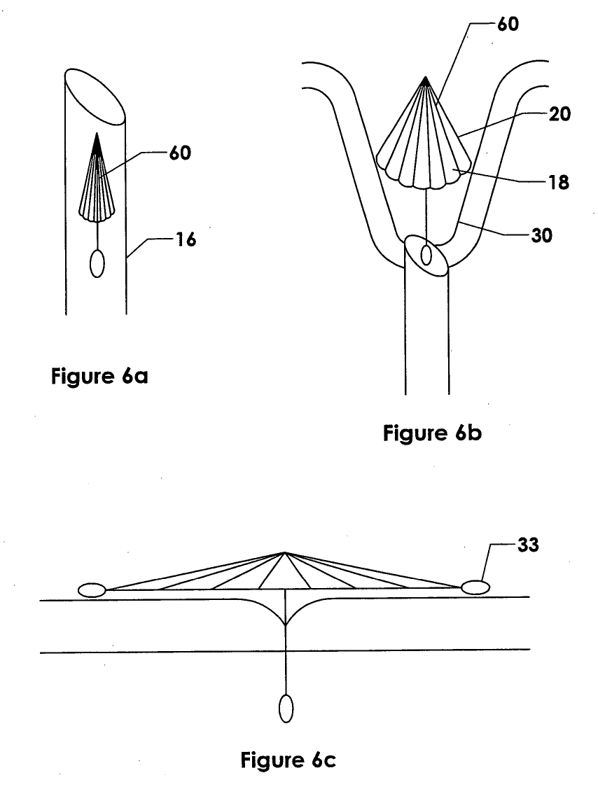

An alternate embodiment of the umbrella anchor is shown in FIG. 6a in which an umbrella 60 is inverted (relative to the configuration of umbrella 14 in FIG. 2a) in the opposite direction inside the tissue penetrating device 16. When deployed as shown in FIG. 6b across the soft tissue, the umbrella 60 unfolds with the mesh 18 and struts 20 pointed toward the outer tissue wall 30. The strut ends 33 can be constructed so that they are rounded and smooth to avoid tissue trauma where they contact the outer tissue wall 30. Furthermore, the struts 20 could be soft with a rounded or flattened surface 33 shown in FIG. 6c so as to distribute any force applied to the center linkage element evenly among them. Alternatively, the strut ends could be sharp with barb elements at the ends to attach securely to the outer tissue wall and prevent lateral migration of the unfolded umbrella.

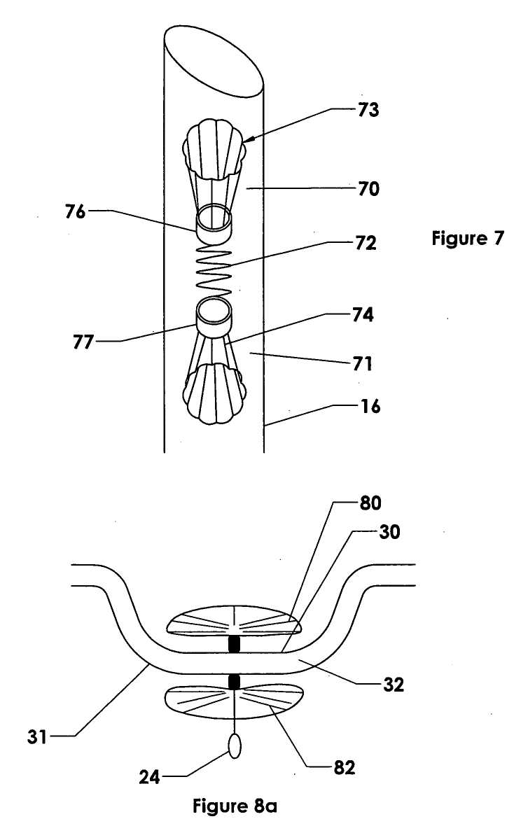

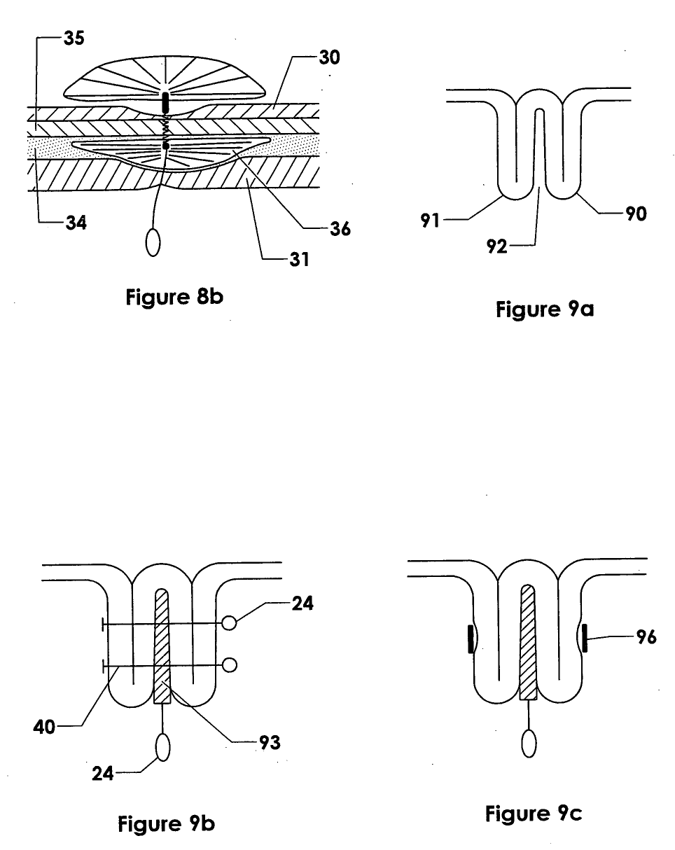

In another embodiment of the invention, an anchoring element is formed by two parallel umbrella structures 70 and 71 that are connected by a center coil spring 72. The two parallel umbrella structures 70 and 71 are shown in FIG. 7 in a collapsed condition inside a tissue penetrating device 16. The umbrellas can be constructed similarly to that described previously with mesh disks 73 and supporting struts 74. The supporting struts 74 of the umbrellas 70 and 71 are coupled to rings 76 and 77 respectively. These rings are connected to the center coil spring 72 located between both umbrellas. The spring 72 is in tension when the rings 76 and 77 are separated and in compression if the rings 76 and 77 are brought closely together. When deployed as shown in FIG. 8a, the upper umbrella 80 is inserted through the soft tissue wall 32 and deployed along the outer tissue wall 30. The lower umbrella 82 is deployed along the inner soft tissue wall 31 so that the soft tissue wall 32 is sandwiched in between these two umbrella structures. The spring 72 which is attached to both umbrellas is in tension in this configuration and provides a constant force along the common axis of the umbrellas to draw each umbrella together. This constant force is intended to secure the umbrellas in place but also to provide a constant pressure between the distal umbrella 80 and the outer soft tissue wall 30. This pressure may force the umbrella and the tissue into contact and promote cell ingrowth into the mesh disk 73. The position of the umbrellas can be further secured to the outer and inner walls of the soft tissue with one or more fastening elements. A linkage element 24 is attached to the center of the lower umbrella 82. Although the lower umbrella 82 is shown in FIG. 8a deployed along the inner soft tissue wall or mucosa 31 of a stomach, alternatively the lower umbrella 82 may be deployed into the submucosa 34 as shown in FIG. 8b. The submucosa 34 does not regenerate and slough off as does the mucosa 31 and may provide a more secure site for deployment of the lower umbrella 82. For deployment into the submucosa 34 a space or bleb 36 may be created by the use of hydro dissection as is commonly done in the art. The lower umbrella 82 may be deployed into the space created by the hydro dissection as shown. Also illustrated are the four predominate layers of soft tissue as found in organs such as the stomach or intestine. The four layers are the serosa 30; which is also referred to in this application as the outer layer of the soft tissue, the muscle wall 35; the submucosa 34 and the mucosa 31; which is also referred to in this application as the inner soft tissue wall.

In several embodiments of the invention that utilize a double-sided anchoring approach such as the parallel umbrella, the proximal element that is deployed either along the inner soft tissue wall 31 or in the submucosa 34 may also serve as a seal between the inside soft tissue wall and the outside soft tissue wall. This seal may help to prevent fluids and bacteria from passing between the two walls. This is important because the outside edge of the serosa 30 is sterile and the inside edge of the mucosa 31 is not.

Another embodiment of the device employs two folds of soft tissue 90 and 91 that are drawn together to create a channel 92 as shown in FIG. 9a. A piece of mesh 93 with a linkage element 24 attached, is inserted into this folded tissue channel 92 and the folds 90 and 91 and the mesh 93 are secured together as illustrated in FIG. 9b. The mesh 93 may be secured to the tissue folds by the use of T-tag fastening elements 40 shown penetrating the folds of tissue and the mesh 93. These fastening elements 40 may also have linkage elements 24 attached. Alternatively, the tissue folds 90 and 91 and the mesh 93 can be captured with a band 96 as shown in FIG. 9c. The band is similar to a rubber band or o-ring and compresses the tissue together securely. The band can be made of elastic materials such as silicone, latex, and deformable materials such as plastic, metal or metal alloys, sutures or fasteners such as tie wraps or various clamps. Other clamp designs are anticipated that provide non-uniform pressure around the circumference of the clamp so as to allow blood flow to the banded tissue. It will be appreciated that the double-folded tissue structure shown in FIGS. 9a-c may be a linear fold, or it may be another shape, such as a circular shape forming a nipple.

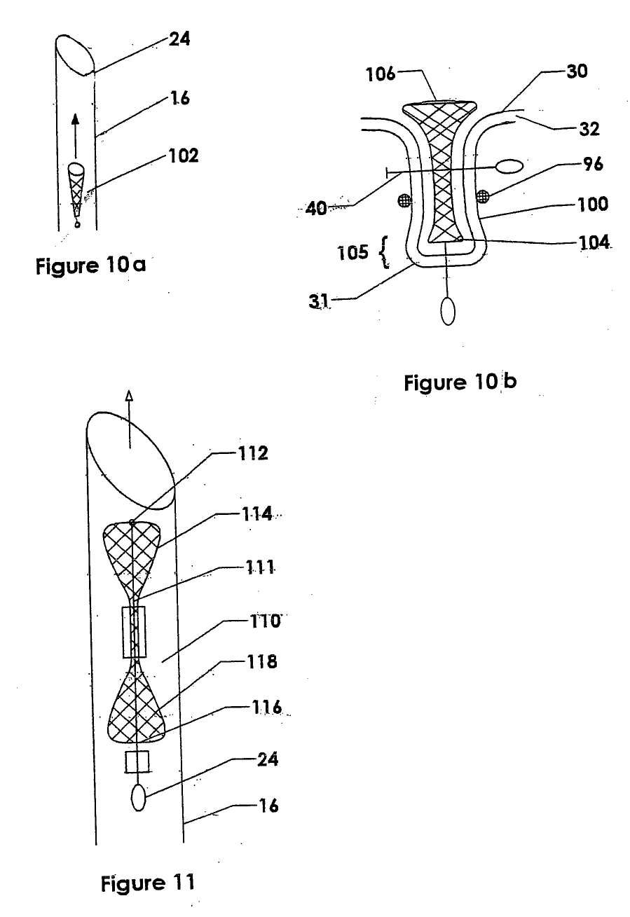

Another embodiment of the device employs a single fold of soft tissue that is drawn together to create a tissue nipple 100 as shown in FIG. 10. This nipple could be formed using the stabilizing element 4 on the end of an endoscope 1. As suction is applied to the working channel 2 of the endoscope, negative pressure is generated inside the cavity of the stabilizing element 4. This causes a tissue nipple 100 to be formed inside the cavity as described previously. A collapsed braided or woven mesh tube 102 is inserted through the soft tissue wall 32 using a tissue penetrating device 16. The mesh tube 102 has a linkage element 24 attached to the proximal end 104 of the mesh tube 102 that remains along the inner wall 31 of the soft tissue. The mesh tube 102 self-expands inside the fold of the outer soft tissue wall 30. The mesh tube 102 may be flared at the distal end 106. The distal end flare 106 is designed to spread out any forces applied to the mesh tube 102 and to facilitate a wide surface area for tissue ingrowth. Likewise the proximal end 104 may employ an enlarged profile that self expands upon deployment. The nipple can be secured by placing fastening elements 40 through the tissue wall 32 and the mesh tube 102 or by attaching a band 96 to the inner wall of the soft tissue as described previously. In all disclosures that utilize a band 96 for securement, the optimal position of the band can be maintained by providing a lip protrusion 105 on the tissue wall. This lip protrusion 105 shown could be formed by the enlarged profile of the mesh tube 102 pushing on the folds of tissue. For example, if the mesh tube 102 has a pre-formed bump at the proximal end 104, when deployed, this bump would force the soft tissue folds to bulge out near the proximal end 104 of the mesh. This bulge in the tissue would provide a lip protrusion 105 that would assist in maintaining the band 96 in position.

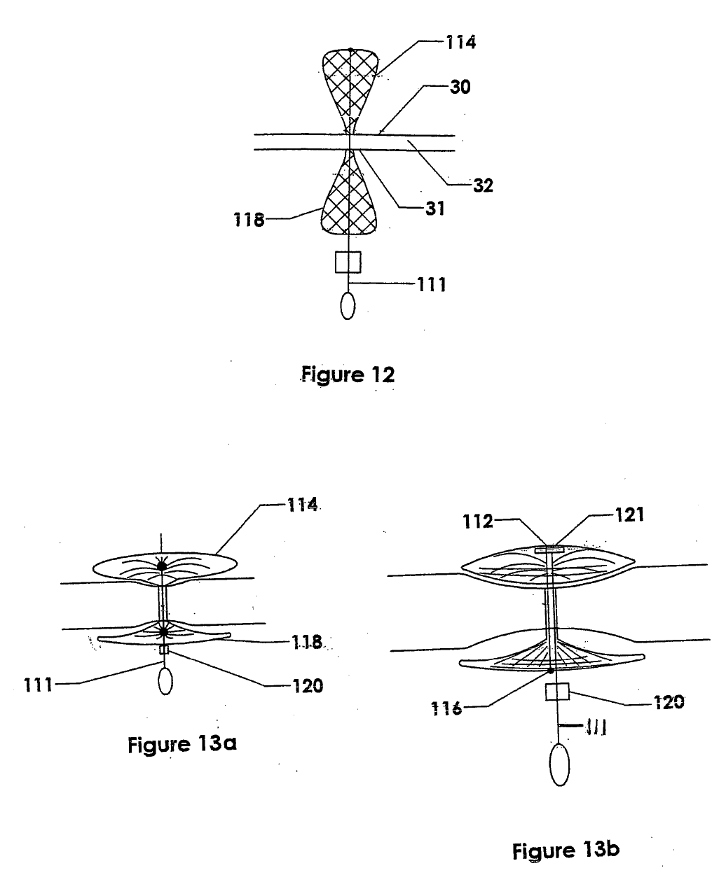

Another embodiment of the soft tissue fastening device is shown in FIG. 11. This embodiment is comprised of two braided mesh balloon shaped wireforms 110 that are coupled together at one end. A collapsing element 111 is connected to the apex 112 of the distal braided mesh wireform 114 and protrudes along the central axis of both wireforms and extends out the apex 116 of the proximal braided wireform 118. A linkage element 24 is attached to the proximal end of the collapsing element 111. Prior to deployment, the wireforms 110 are collapsed inside the tissue penetrating device 16. Upon deployment as shown in FIG. 12 the distal wireform 114 is inserted through the soft tissue wall 32 and positioned along the outer wall or serosa 30 and the proximal wireform 118 is deployed along the inner wall or mucosa 31 so that the soft tissue wall 32 is sandwiched between these two collapsed braided mesh wireforms. Once in position across the tissue wall, the collapsing element 111 is pulled which causes the two braided mesh balloon shaped wireforms 114 and 118 to expand into a flat mushroom shape as shown in FIG. 13a. The position of the collapsing element 111 may be secured in position with a knot or cinching element 120 such as a wire band, ferrule, suture or a tension member. The cinching element may be any of many well described suture or rope cinching devices that are known in the art or publicly described for other applications. Alternatively the proximal wireform 118 may be deployed into the submucosa 34 utilizing techniques described previously and shown by way of example in FIG. 8b.

The use of the cinching element 120 may also keep a slight tension on the collapsing element 111. It is intended that this tension will maintain a slight pressure between the distal wireform 114 and the outer soft tissue wall 30 to promote cell ingrowth into the distal wireform 114. Although this pressure is maintained by tension on the collapsing element 111 in this embodiment, it is anticipated that many of the embodiments in this invention may utilize other means of maintaining at least a temporary tension in the anchor system. Such tension may be provided by an externally applied force through the use of a cinching member or a secondary attachment such as a T-tag. Such tension may also be provided by an internal force such as a spring member or other pre-loaded tension means that are designed to secure the anchor to the soft tissue but also to provide a slight pressure between the device and outer soft tissue wall 30.

The collapsing element 111 may also be configured to provide additional function in the design. In this alternate embodiment shown in FIG. 13b the collapsing element 111 has two ends; one terminates in the linkage element 24 and the other is secured to the apex 116 of the proximal braided wireform 118. The collapsing element 111 is routed from the apex 116 through the center of the wireforms to the apex 112 of the distal braided mesh wireform 114. At this point the collapsing element 111 loops around a strut or pulley 121 located at apex 112 and passes back down along the center of both wireforms through the apex 116 of the proximal wireform. In this configuration as the linkage element is pulled, the two wireform apices are drawn together expanding their diameters and creating a tension between the two wireforms. The collapsing element 111 may be secured in position and thus the tension maintained with a cinching element 120 such as a wire band, ferrule, suture or a tension member. This tension can be beneficial to maintain the expanded state of the wireforms and to promote cell ingrowth into the distal wireform. This tension force may also be applied as the anchor is utilized by secondary devices that place a load on the linkage element 24. As the linkage element 24 is pulled by the secondary devices, a tension force is applied to the collapsing element which draws the two wireform shapes together. This configuration of collapsing element described may be utilized in many of the other embodiments that utilize a central pull wire or collapsing element to collapse the anchor length and expand its diameter.

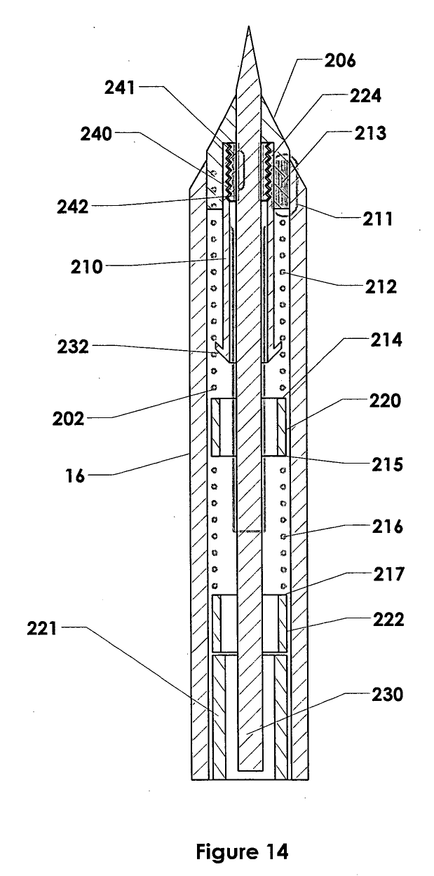

An alternate embodiment of the invention that expands further upon the mesh wireform concept is shown inside a tissue penetrating device shown in FIG. 14. The tissue penetration device 16 is pre-loaded with a wireform assembly 202 prior to placement into the working channel of an endoscope. The wireform assembly 202 is comprised of a sealing cap 206, a retainer 210, two wireforms 212 and 216, a joining band 220, a pusher tube 221 and a needle 230. The sealing cap 206, the retainer 210 and the distal end 213 of the distal wireform 212 form a distal assembly 211 and are joined together at a distal joint 224. The components of this distal assembly 211 may be fused, welded, soldered, heat bonded, glued or attached using various methods known in the art. The needle attachment element 240 and the needle 230 form a needle assembly 241 and are joined together at distal joint 242. Likewise this needle assembly 241 may be fused, heat bonded, glued or attached using various methods known in the art. The needle assembly 241 can be connected to or disconnected from the distal assembly 211 of the wireform assembly 202 by the operator. This connection and disconnection can be accomplished through the use of thread joints, locking and unlocking tabs, bayonet connections, conical taper joints or any number of mechanical latching mechanisms. The examples here are not meant to be a comprehensive list and any mechanical connection mechanism known in the art would be acceptable. The view of FIG. 14 illustrates only one such concept and utilizes a threaded joint to connect the needle assembly 241 to the distal assembly 211. In this example the assemblies are shown connected and can be disconnected by rotating the proximal end of the needle 230 which extends down the inside of the tissue penetrating device 16 and out from the proximal end of the endoscope (not shown). Other connection means would likewise be detachably connected by the operator by manipulating the proximal end of the needle 230 from outside the patient.

The two wireforms 212 and 216 are joined together with the joining band 220. The proximal end 214 of wireform 212 and the distal end 215 of wireform 216 are joined together with the joining band 220 using various means such as fusing, soldering, welding, gluing or can be joined using various other methods known in the art. The proximal end 217 of wireform 216 is joined to the proximal band 222 using various means such as fusing, soldering, welding, gluing or can be joined using various other methods known in the art. The pusher tube 221 is preferably a hypodermic tube and extends through the inside of the tissue penetrating device 16 and out from the proximal end of the endoscope (not shown).

The wireform assembly 202 is initially placed in the tissue penetrating device 16 with the two wireforms 212 and 216 in a collapsed position and the needle assembly 241 connected to the distal assembly 211 such that the needle 230 protrudes from the sealing cap 206 as shown in FIG. 14. The pusher tube 221 is used to advance the wireform assembly 202 until the needle 230 and sealing cap 206 slightly protrude from the end of the tissue penetrating device 16.

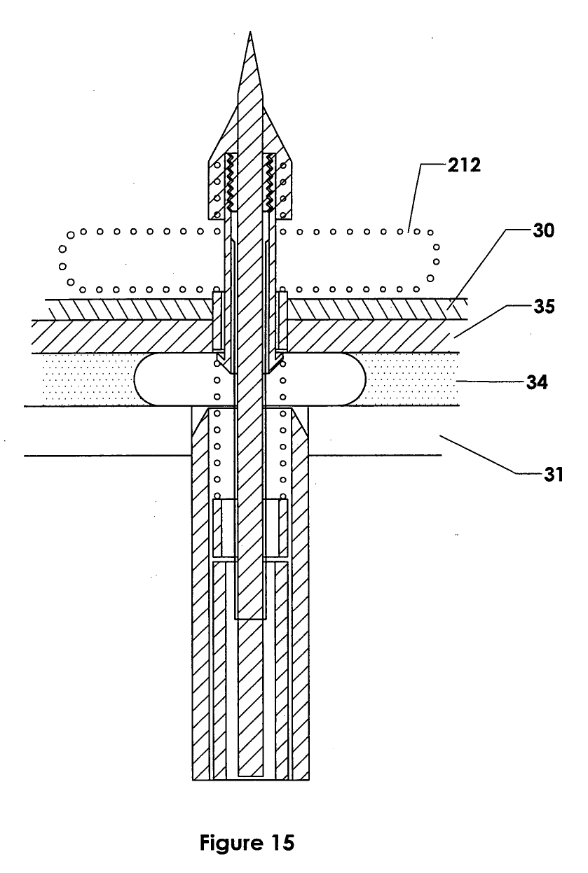

The tissue penetrating device 16 is preferably preloaded inside the working channel of an endoscope. The endoscope is brought to the site of the intervention and the tissue penetrating device 16 and wireform assembly 202 are advanced through the soft tissue wall. The pusher tube is advanced which forces the wireform assembly 202 out from the tissue penetrating device 16. As the assembly 202 advances, the distal wireform 212 is released from the restraining inside walls of the tissue penetrating device 16 and self-expands to a new larger diameter as shown in FIG. 15. As the distal wireform 212 is expanded the proximal band 220 moves past the locking tabs 232 of the retainer 210. The locking tabs 232 have an angled end that prevents the proximal band 220 and the distal wireform 212 from reversing direction and re-assuming a collapsed condition.

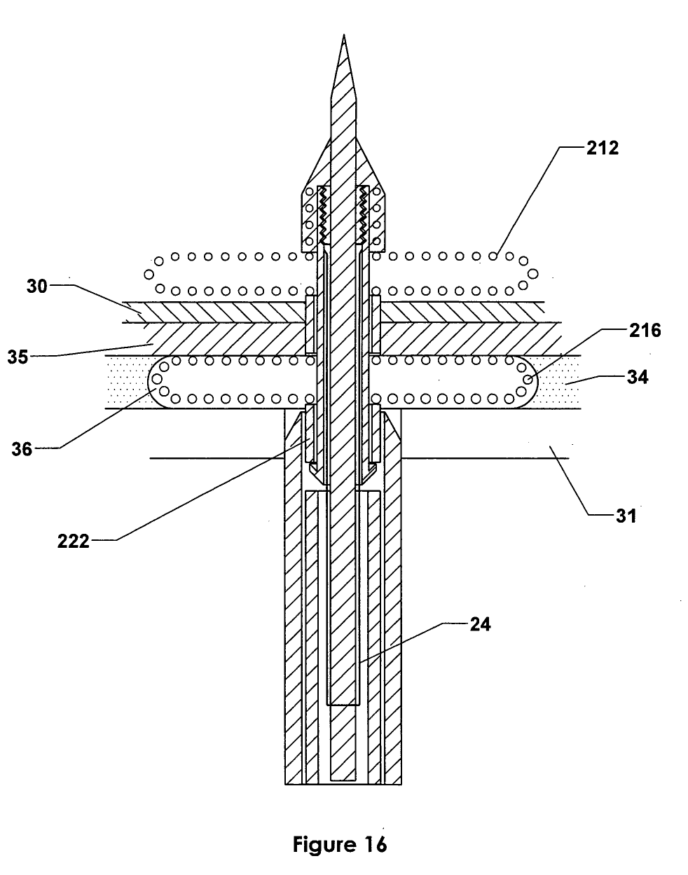

Once the distal wireform 212 is expanded on the outside wall of the soft tissue 30, the, proximal wireform 216 can be deployed. This wireform may be deployed along the inside soft tissue wall 31 (not shown) or into the submucosa 34 of the soft tissue wall. Before the later condition can be accomplished, a cavity or bleb 36 may be formed with this device by injecting fluid into the proximal end of the pusher tub 221. This fluid can be directed to exit the tissue penetrating device 16 through the coils of the collapsed proximal wireform 216 as shown in FIG. 15. The fluid may create a cavity by hydrodissection in the submucosa 34 of the soft tissue wall. Once this cavity is created, the proximal wireform 216 is advanced using the pusher tube 221 and the proximal wireform 216 expands to new larger diameter inside the submucosa 34. As the proximal wireform 216 is advanced, the proximal wireform joint 222 moves past the locking tabs 232 of the retainer 210. These tabs prevent the proximal wireform joint 222 and the proximal wireform 216 from reversing direction and re-assuming a collapsed condition as shown in FIG. 16. Once both wireforms are expanded and properly positioned, the needle assembly 241 can be disconnected from the distal assembly 211 and removed by rotating the needle and unscrewing the threads. As the needle 230 is removed from the sealing cap 206, the sealing cap 206 closes to seal the opening. This prevents any fluid communication between the outer soft tissue wall 30 and the inner tissue wall or mucosa 31. A linkage element 24 is attached to the wireform assembly 202 and is positioned inside the inner wall of the soft tissue once the tissue penetrating device 16 is removed.

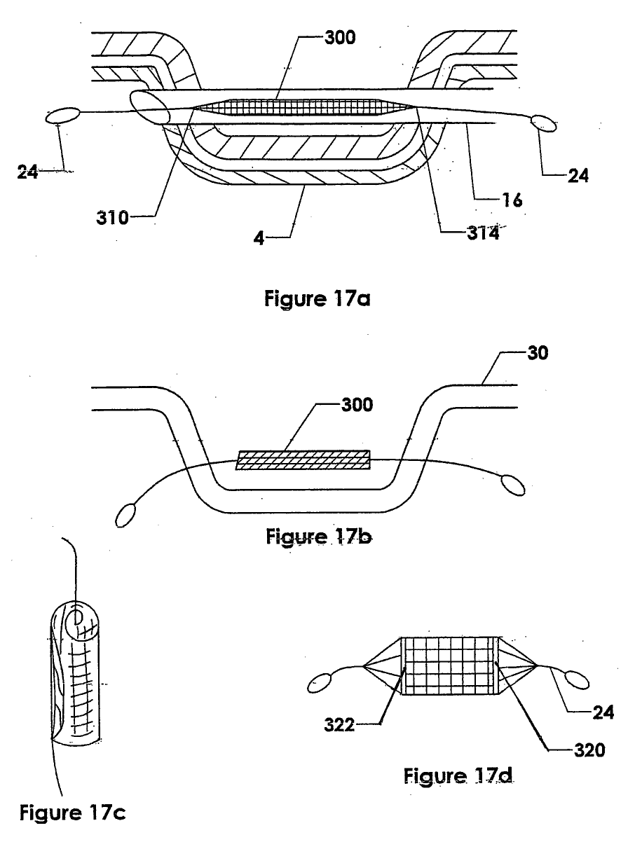

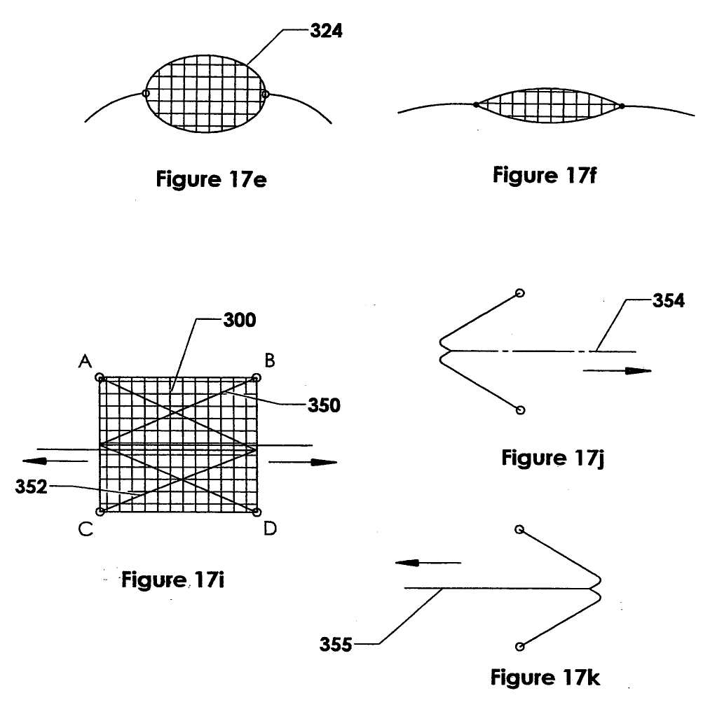

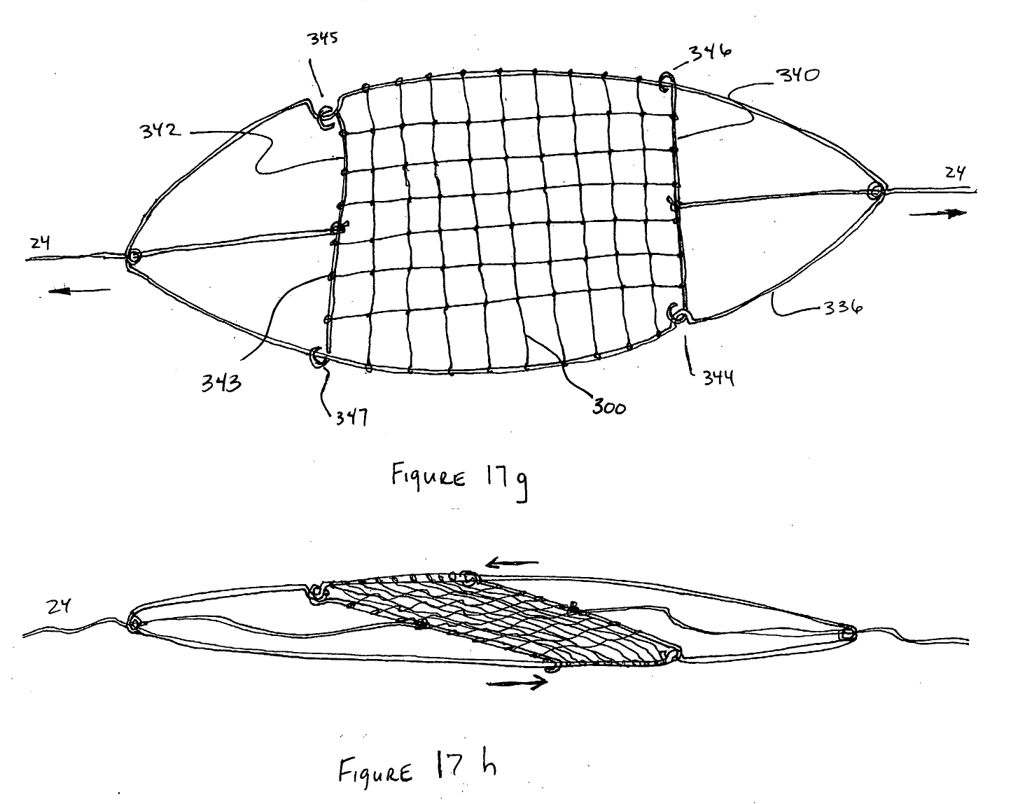

Still another embodiment of the invention is a hammock type anchor 300 that can be deployed by a tissue penetrating device 16. The walls of the soft tissue may be first drawn into the cavity of a stabilizing element 4. The tissue penetrating device 16 penetrates through two walls of soft tissue as shown in FIG. 17a. A hammock 300 is formed as a mesh or from material that has been described previously. The hammock 300 is collapsed or rolled up inside the penetrating device 16. The hammock 300 has linkage elements 24 attached to the distal 310 and proximal 314 ends of the hammock. The hammock 300 is pushed out until the linkage element at 310 protrudes through the tissue wall at a first location and then the tissue penetrating device 16 is withdrawn leaving the hammock 300 situated along the inner soft tissue wall or serosa 30 and a linkage element at the proximal end 314 of the hammock 300 protruding through the tissue wall at a second location as shown in FIG. 17b.

Various hammock designs or features are described in FIGS. 17c-17k. The hammock may be rolled up as shown in FIG. 17c for insertion into a tissue penetrating device. The hammock 300 may also utilize distending members 320 and 322 as shown in FIG. 17d. These distending members are designed to collapse so that the hammock may be passed through a tissue penetrating device and then self expand and spread out the hammock 300 when deployed. The distending members 320 and 322 may be made from metal or metal alloys such as stainless steel, Nitinol or Elgiloy or from plastics such as PTFE, delrin, polyolefin, nylon or polyether. These members support the ends of the mesh, are attached at each end to the linkage elements 24 and spread out any force that is applied to the linkage element 24 along their length. These members also insure that the hammock mesh will be deployed with a large surface area. This design also minimizes the area of the semi rigid elements that may be in contact with the soft tissue which may be beneficial to inhibit pull out of the anchor. The hammock mesh may be encircled by a frame 324 made from a material such as Nitinol as shown in FIG. 17e. This superelastic frame could collapse for introduction as shown in FIG. 17f and then expand to a larger diameter when released from the penetrating device 16.

Another embodiment of the distending members is displayed in FIGS. 17g and 17h. The mesh hammock 300 is supported by the frame 336 and distending struts 340 and 342. The mesh hammock 300 is loosely coupled to distending struts 340 and 342 and the frame 336 at various points 343. The distending struts 340 and 342 are coupled to the frame 336 at pivot points 344 and 345 and at sliding points 346 and 347. The pivot points 344 and 345 are created by forming small indentations in the frame 336. The distending struts 340 and 342 are also attached to the linkage elements 24. The mesh hammock 300 can be collapsed for deployment through a tissue penetrating device by pivoting the distending members 340 and 342 about pivot points 344 and 345 and sliding the sliding points 346 and 347 in the directions of the arrows shown in FIG. 17h. The assembly can be collapsed for introduction and then deployed by pulling the linkage elements in opposite directions as shown by the arrows in FIG. 17g.

Another embodiment of the distending members is displayed in FIG. 17i. The mesh hammock 300 is attached to two oppositely formed Nitinol shapes 350 and 352 at four points A, B, C&D on the mesh. The Nitinol shapes are shown in FIGS. 17j and 17k. The Nitinol shapes are attached to sutures 354 and 355 which are in turn attached to linkage elements 24 (not shown). The hammock is initially collapsed inside a penetration device and then the sutures are pulled in opposite directions to deploy the hammock.

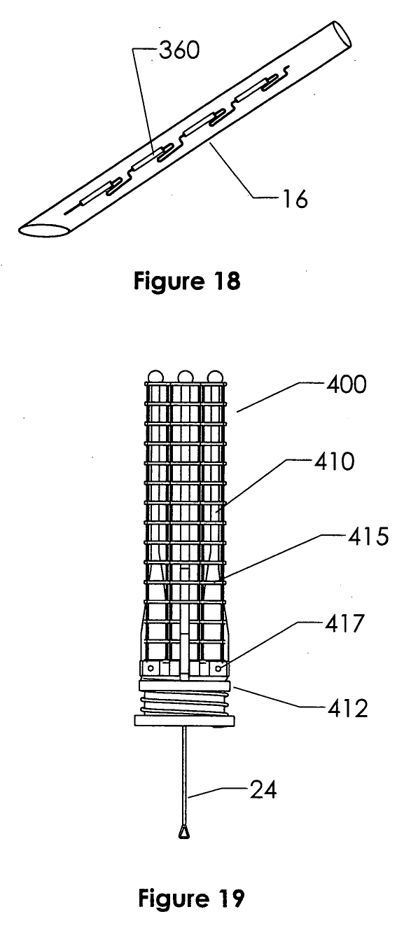

Many of the securement anchors thus described may be secured individually or may be also possible to secure more than one anchor as a group. FIG. 18 shows a series of anchor devices contained inside the inner lumen of a tissue penetrating device 16. Often more than one anchor is useful for a procedure and the ability to deploy more than one anchor without the need to withdraw the tissue penetrating device may be advantageous. The multiple anchor devices contained inside the penetrating device 16 may be serially connected by suture 360 and deployed as a continuous chain of anchors as shown, or in pairs or other combinations. Alternatively the multiple anchor devices may be loaded individually into the penetration device and deployed as individual anchors that could be placed individually.

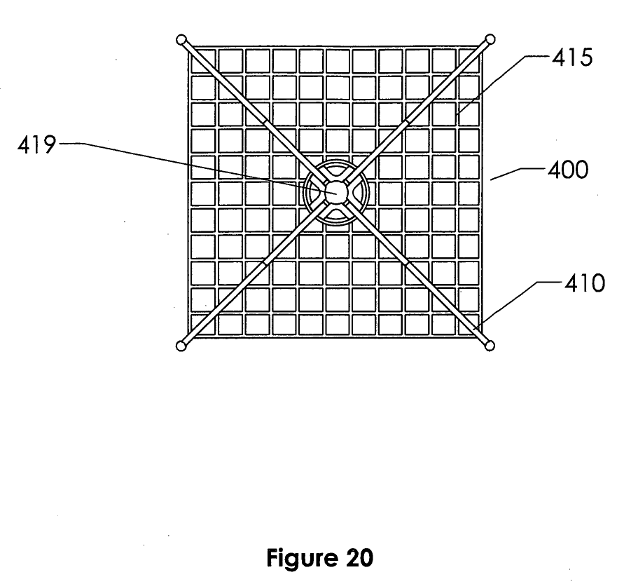

Another embodiment of an anchor element that utilizes a tissue interface 400 with foldable struts is illustrated in FIGS. 19 and 20. The tissue interface 400 is shown in a collapsed condition in FIG. 19 and can be delivered with the use of a delivery device which can be the working channel of an endoscope or a tissue penetrating device 16. The tissue interface 400 is comprised of a mesh element 415 and at least one strut 410 that is hingely attached to a central hub 412. The strut is attached to the mesh element 415 that can be made of material suitable for promoting tissue ingrowth as described previously. The mesh element 415 is also designed so that it can collapse into a small volume suitable for loading into a delivery device. As shown, the mesh element 415 is collapsed either alongside the strut 410 or within a diameter created by the strut 410. In the delivery device, the strut 410 lies parallel to the main axis of the delivery device.

The tissue interface 400 is deployed by pushing it out from a delivery device and causing the tissue interface 400 to unfurl along the outer wall of the soft tissue. As the tissue interface 400 exits the delivery device, the constraining forces of the delivery device are removed and the strut 410 either self-rotates about a pivot point 417 or can be manually directed to unfold until the strut 410 resides in a fully deployed condition as shown in FIG. 20. In this condition, the strut 410 is configured to be essentially perpendicular to the axis of the delivery device. However the tissue interface 400 can be deployed at various angles between, for example, 30° and 120° to the axis of the delivery device. A linkage element 24 is attached to the bottom of the central hub 412 and will extend through the soft tissue wall 32 when the tissue interface 400 is deployed.

In one embodiment the struts 410 could be spring loaded such that as they exit the delivery device they unfold and snap into position. In another embodiment the struts 410 could have pull-wires attached that extend through the delivery device. These pull-wires could be activated by the operator to cause the struts 410 to rotate for deployment. The struts 410 could also be deployed utilizing one or more inflatable balloons. These balloons could be positioned inside the mesh/strut assembly and be inflated by the operator and then deflated once the struts 410 were in proper position.

When the tissue interface 400 is deployed as illustrated in FIG. 20, the struts 410 form a framework that provides structural integrity to the mesh element 415. As loads are applied to the center 419 of the central hub 412, the force is distributed along the struts 410 and ultimately to the mesh element 415 which is in direct contact with the outer soft tissue wall 30 or the serosa. The mesh element 415 can be rigidly attached at one or more points along the strut 410 or the mesh element 415 can be loosely attached at one or more points along the strut 410. A loose attachment configuration might permit more freedom of movement and thus permit the mesh to move as needed. As the strut 410 unfolds, it causes the mesh element 415 to be unfolded into a configuration whereby the mesh element exposes a maximal surface area to the soft tissue. The mesh element 415 is preferably stretched tight by the strut 410 deployment but the mesh element 415 may also be loosely deployed.

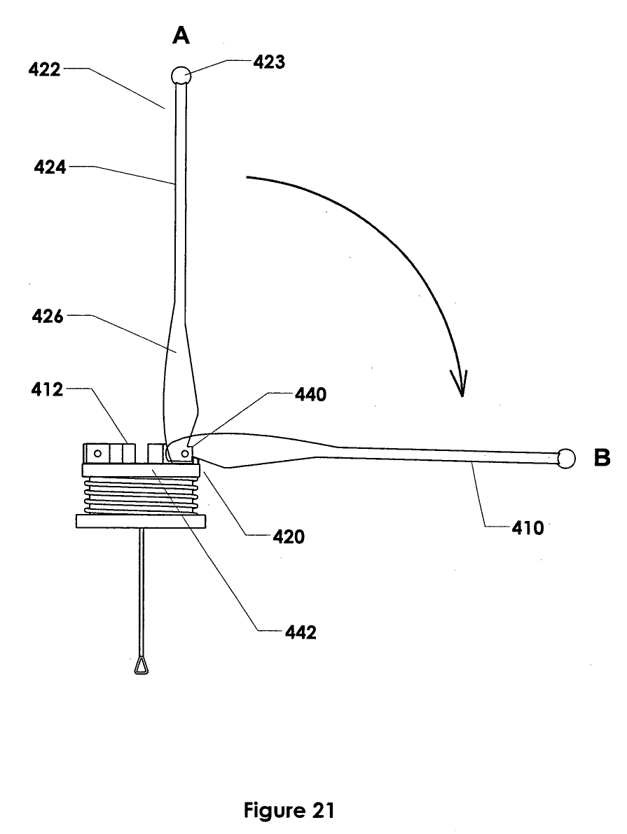

Referring to FIG. 21, the strut 410 is hingedly attached to the central hub 412 and has an internal end 420 and an external end 422. The external end 422 terminates the strut 410 and can be the outermost mesh element 415 attachment point. The end of the strut 423 is smooth and rounded so as to avoid damage to the soft tissue surrounding it. Similarly, the strut body 424 is intended to be rigid enough to transmit force along its length but also flexible enough to avoid damage to surrounding tissue. The internal end 420 of the strut 410 terminates in a connector 426 that is integrally formed with the strut body 424. In another embodiment the connector 426 may be slideably attached to the strut body 424 such that the strut 410 may move relative to the connector 426. This ability of the strut 410 to move may be beneficial in order to accommodate natural expansion and contraction of the soft tissue, as in the case of a stomach wall, or also to accommodate growth of tissue into the mesh element 415. This tissue growth may exert forces on the tissue interface 400 and require it to be flexible and alter its shape from the original deployed configuration. In still another embodiment the connector 426, the strut body 424 and the central hub 412 may be made from bioabsorbable materials that decouple and are absorbed by the patient's body over time. As shown by the arrow, the strut 410 can be in a first position A such that the strut 410 is aligned with the axis of the delivery device for introduction into the body. The strut 410 can rotate about the pivot point 417 in the direction of the arrow and move to a second position B. In this position the strut is deployed and the connector 426 is locked into position.

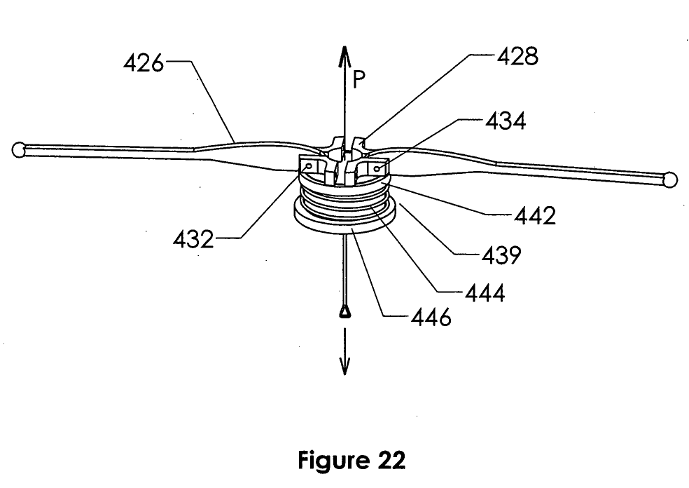

Referring now to FIGS. 19-22, it can be seen that the connector 426 is connected to one or more strut engagement elements 428 positioned at the upper end of the central hub 412 with a pivot pin 417 that is placed through the holes 434 of the strut engagement element 428 and connector 426. The pivot pin 417 may also be made of bioabsorbable materials that are reabsorbed by the patient's body over time which facilitates the decoupling of the connector 426 and strut engagement elements 428. In this situation, the mesh element 415 may or may not remain coupled to the central hub. The lower portion of the central hub 412 has a strut locking mechanism 439 that is comprised of a locking ring 442, spring 444 and ledge 446. The spring 444 is a coil spring captured between the ledge 446 and the locking ring 442. The spring exerts an outward force on the locking ring 442 and the ledge 446 that pushes these two elements away from each other. Other types of springs such as leaf springs, compression springs, and o-rings could also be employed and the use of alternate spring types is anticipated. The ledge 446 and the strut engagement element 428 are coupled together. The locking ring 442 is fitted around the ledge and is able to move along the primary axis P of the assembly. As the locking ring 442 is moved toward the ledge 446 in the opposite direction of P, the spring 444 is compressed. As the locking ring 442 is moved away from the ledge 446, the spring 444 is expanded. The connector 426 is shaped such that as it is rotated about the pivot pin 417, force is exerted on the locking ring 442 and it is moved toward the ledge 446 which in turn compresses the spring 444. This action exerts an opposite force against the connector 426. As the connector 426 continues to rotate about the pivot pin 417, a latch element 440 formed in the connector 426 engages the locking ring 442. Once the locking ring 442 is positioned in the latch element 440, as shown in FIG. 21, the connector 426 is locked into position. The strut securement of this type may be important to create a semi-rigid structure that is capable of distributing forces from the linkage element 24 to the center of the central hub 419 and then to the mesh element 415.

Although this invention has been disclosed in the context of certain preferred embodiments and examples, it will be understood by those skilled in the art that the present invention extends beyond the specifically disclosed embodiments and/or uses of the invention and obvious modifications and equivalents thereof Thus it is intended that the scope of the present invention herein should not be limited by the particular disclosed embodiments described above, but should be determined only by a fair reading of the claims that follow.

What is claimed is:

1. A device for securing to a soft tissue wall comprising:
an umbrella-shaped anchor element having a central hub;
supporting struts coupled to and radiating from said hub;
a tissue interface disposed over the struts;
the anchor element capable of self-expanding from a first collapsed configuration to a second or expanded radially outward configuration.

2. The tissue interface of claim 1 wherein the tissue interface is a membrane.

3. The tissue interface of claim 1 wherein the tissue interface is a mesh having sufficient porosity to facilitate cellular ingrowth or attachment.

4. The tissue interface of claim 3 wherein the mesh element is made of wire, fabric or plastic that is woven or braided.

5. The tissue interface of claim 3 having a coating on a side adjacent to the tissue wall which has cellular growth promoting properties.

6. The struts of claim 1 wherein the struts are made from shape memory alloys.

7. The tissue interface of claim 2 having a coating on a side opposite the tissue wall which has cellular growth or cellular attachment inhibiting properties.

8. The tissue securement device of claim 3 wherein the radially expanding anchor element is positioned inside a portion of the soft tissue wall.

9. The tissue device of claim 1 further comprising at least one fastening element which passes through the tissue wall and secures the tissue interface to the tissue wall at a location spaced apart from the central hub.

10. The anchor of claim 1 wherein the struts are capable of locking in position once the struts have expanded to a radially outward configuration.

11. The anchor of claim 10 wherein the struts are capable of distributing the force of a load applied to the central hub across the surface area of the tissue interface.

12. A device for securing to a tissue surface having an inner and an outer wall comprising:
a first umbrella-anchor element having a first central hub;
supporting struts coupled to and radiating from said hub;
a first tissue interface disposed over the struts;
a second umbrella-anchor element having a second central hub;
supporting struts coupled to and radiating from said hub;
a second tissue interface disposed over the strut;
the anchor elements capable of self-expanding from a collapsed configuration to an expanded radially outward configuration;
the anchor elements each coupled to a spring; the spring configured to draw the hubs together thereby sandwiching the soft tissue in between the anchors.

13. The tissue interfaces of claim 12 further comprising mesh elements having sufficient porosity to facilitate cellular ingrowth or attachment.

14. The tissue interfaces of claim 13 wherein the mesh elements are made of wire, fabric or plastic.

15. The mesh elements of claim 14 wherein the mesh is woven or braided.

16. The distal tissue interface of claim 13 having a coating on the side adjacent to the outer tissue wall which has cellular growth promoting properties.

17. The distal tissue interface of claim 13 having a coating on the side opposite the outer tissue wall which has cellular growth or cellular attachment inhibiting properties.

18. The tissue securement system of claim 12 further comprising at least one fastening element which passes through the first tissue interface, the tissue wall and the second tissue interface and secures the tissue interfaces to the tissue wall and each other at a location spaced apart from the first and second central hubs.

19. The anchors of claim 12 wherein the struts are capable of locking in position once the struts have expanded to a radially outward configuration.

20. A device for securing to a tissue surface having an inner and an outer wall comprising:
proximal and a distal mushroom-shaped mesh wireforms that are coupled together end to end;
a collapsing element routed along the axis of the wireforms and connected to an apex of the distal wireform;
wherein as the collapsing element is pulled, the wireforms change from an axially collapsed configuration to a second or expanded radially outward configuration.

21. The device of claim 20 wherein the proximal mushroom-shaped wireform is positioned along the inner wall and the distal mushroom-shaped wireform is positioned along the outer wall when the device is in an expanded radially outward configuration thereby sandwiching the tissue in between.

22. The device of claim 21 further comprising a cinching element that can be attached to the collapsing element to maintain the expanded configuration of the wireforms.

23. The device of claim 22 further comprising a linkage element coupled to the end of the collapsing element.

24. The mesh wireform of claim 21 wherein the mesh is made of wire, fabric or plastic.

25. The mesh elements of claim 24 wherein the mesh is woven or braided.

26. The mesh wireform of claim 21 having a coating on the side adjacent to the outer tissue wall which has cellular growth promoting properties.

27. The device of claim 21 further comprising at least one fastening element which passes through the proximal mushroom-shaped wireform, the tissue and the distal mushroom-shaped wireform and secures the mushroom-shaped wireforms to the tissue and each other at a location spaced apart from the axis of the wireforms.

28. The device of claim 20 further comprising the apex of the distal wireform having a pulley; the collapsing element attached to an apex of the proximal wireform and routed along the axis of the wireforms through the pulley and back along the axis of the wireforms such that as the collapsing element is pulled the wireforms change from a collapsed configuration to a second or expanded radially outward configuration.

29. A device for securing to a tissue surface comprising:
a mesh hammock having linkage elements positioned at each end;
at least one distending member coupled to the hammock such that as the hammock unfolds from a delivery configuration to an expanded anchoring configuration;
the distending member spreads out the surface area of the hammock.

30. The device of claim 29 further comprising a single piece distending member that encircles the hammock as a frame.

31. The device of claim 30 wherein the distending member is made from made from metal or metal alloys such as stainless steel, Nitinol or Elgiloy or from plastics such as PTFE, delrin, polyolefin, nylon or polyether.

32. The device of claim 29 further comprising a mesh hammock encircled by a frame and loosely coupled to the frame and two distending members;
opposite ends of the distending members coupled to the frame;
the other ends of the distending members slideably attached to the frame; and linkage elements connected to the center of the distending members such that as the linkage elements are pulled in opposite directions, the hammock unfolds from a collapsed configuration to an expanded anchoring configuration.

33. A method of placing an anchor into tissue having an inner and an outer wall comprising: placing a collapsed first umbrella-shaped anchor element, having a central hub, supporting struts coupled to and radiating from said hub, a linkage element attached to the hub and a tissue interface disposed over the struts, through the tissue; unfolding the umbrella-shaped anchor such that the open end of the umbrella faces the outer tissue wall; and the linkage element extending through the tissue to the inner wall.

34. The method of claim 35 further comprising a second umbrella-shaped anchor element, having a central hub, supporting struts coupled to and radiating from said hub, a tissue interface disposed over the struts, and the second umbrella-shaped anchor element attached to the first umbrella-shaped anchor element end to end; wherein each anchor element is deployed on opposing tissue walls; and the linkage element extends through the wall to an inside portion of the wall;
and providing a force between the two anchor elements to draw the umbrella-shaped anchors together sandwiching the soft tissue in between.

35. The method of claim 34 further comprising placing at least one fastening element through the second umbrella-shaped anchor element, the tissue and the first umbrella-shaped anchor element; and securing the anchor elements and the tissue together at a location spaced apart from the central hubs.

36. A method of placing an anchor into tissue having an inner and an outer wall comprising:
placing an anchor element comprising a collapsed proximal and distal mushroom-shaped mesh wireforms that are coupled together end to end, into the tissue;
the anchor element having a collapsing element routed along the axis of the wireforms and connected to an apex of the distal wireform;
pulling the collapsing element to cause the wireforms to change from an axially collapsed configuration to a second or expanded radially outward configuration with one wireform along each tissue wall, sandwiching the tissue in between.
